(19) United States
(12) Patent Application Publication
(10) Pub. No.: US 2005/0043597 A1
(43) Pub. Date: Feb. 24, 2005

(54) Optical vivo probe of analyte concentration within the sterile matrix under the human nail

(75) Inventor: Jinchun Xie, Cupertino, CA (US)

Correspondence Address: Michael D. Van Loy;Skymoon Research and Development, LLC, Intellectual Property Department, 3045 Park Boulevard, Palo Alto, CA, 94306, US

(73) Assignee: Skymoon Research and Development, LLC

(21) Appl. No.: 10/723,042

(22) Filed: Nov. 26, 2003

Related U.S. Application Data
(60) Provisional application No. 60/491,367, filed on Jul. 31, 2003.

(51) Int. Cl. A61B 5/00

(52) U.S. Cl. 600/315

(57) ABSTRACT

Method and systems are provided for in vivo, non-invasive detection of blood analytes. A portion of the sterile matrix located beneath a nail is illuminated by passing radiation from an optical source through the nail into the sterile matrix. Scattered, refracted, or reflected radiation emitted within the sampled volume is collected and analyzed to identify and quantify one or more selected analytes.

### FIELD OF THE INVENTION

The present invention provides a process for non-invasive, in vivo optical detection of analytes, such as for example, glucose, by optically probing the sterile matrix located underneath a nail, such as for example, a fingernail or a toenail. The sterile matrix may be probed using Stokes Raman spectroscopy, although other optical probe techniques can also be employed, including, but not limited to, near infra-red (NIR) reflective absorption spectroscopy and optical coherence tomography.

### BACKGROUND OF THE INVENTION

There has long been considerable interest in the non-invasive monitoring of body chemistry. For example, there are approximately 16 million American diabetics. World wide, more than 100 million diabetics are advised to monitor their glucose levels several times each day. Using currently available methods for measuring blood glucose levels, many diabetics must give blood five to seven times per day to adequately monitor their insulin requirements. The vast majority of diabetics would greatly benefit from a simple and accurate method for the non-invasive measurement of blood glucose levels. With a non-invasive blood glucose measurement procedure, closer control of glucose levels could be achieved and the continuing damage, impairment, and costs caused by diabetes could be dramatically reduced. In addition, there is a great interest in an optical measurement technique that would permit simultaneous analysis of multiple other components (analytes) present in whole blood without the need for complex conventional sample processing techniques, that typically involve drawing blood followed by centrifuging and/or adding multiple reagents. Other analytes of interest in addition to glucose include, but are not limited to, urea, cholesterol, triglycerides, total protein, albumin, hemoglobin, hematocrit, and bilirubin. However, optical analysis of whole blood is complicated by the presence of many target analytes in low concentration. The weak signals resulting from such low concentrations may be further distorted by absorption and scattering caused by red blood cells and/or other components of living tissue.

Raman scattering describes the phenomenon whereby incident light scattered by a molecule is shifted in wavelength from the incident wavelength. The magnitude of the wavelength shift depends on the vibrational motions the molecule is capable of undergoing, and this wavelength shift provides a sensitive measure of molecular structure. That portion of the scattered radiation having shorter wavelengths than the incident light is referred to as anti-Stokes scattering, and the scattered light having wavelengths longer than the incident beam as Stokes scattering.

The use of Raman spectroscopy in the biological sciences has heretofore suffered from two major obstacles. One is the strong fluorescence caused by the incident light manifested by the majority of the biological molecules being investigated and/or by impurities present in them. The fluorescence process is inherently more probable than Raman scattering. Thus, the intensity of fluorescence emissions tends to overshadow weaker Raman signals. Photodecomposition of tissue by incident light may also create another strong fluorescence source that presents an additional obstacle to in vivo spectroscopic measurements. Fluorescence from most biological materials tends to be less intense in the visible and near infra-red (NIR) spectral regions. Use of NIR spectroscopic incident light may also reduce photo-decomposition and/or photo induced transformation of tissue samples and biological analytes.

Light scattering may be classified as elastic or inelastic scattering. Elastic scattering changes the direction of light propagation but not the light energy (i.e. the frequency or wavelength of the incident light). The causes of elastic scattering include rough surfaces or index mismatched particles as well as Rayleigh scattering from molecules. Inelastic scattering from matter changes the light energy as well as the propagation direction and matter, and is called Raman scattering. Raman scattering is a very powerful spectroscopic method for the detection of analytes, as the Raman spectra of different analytes are frequently more distinct than the spectra obtained by direct light absorption or reflectance. Although Raman spectroscopy has heretofore been suggested as a means to non-invasively monitor blood glucose concentration, human tissue generally causes strong elastic scattering of light, which makes illumination of suitable blood-containing tissues difficult and also complicates the collection of Raman (inelastic) scattered radiation. For non-invasive detection of glucose or other analytes present in the blood, incident laser radiation cannot generally reach tissue filled with blood capillaries without passing through the skin. Because skin generally contains numerous species, such as for example melanin and other pigmentation that absorb and/or scatter incident light, spectroscopic analysis though the skin is problematic. As such, development an improved system and method for in vivo detection and quantification of blood an/or tissue analytes is highly desirable.

### SUMMARY OF THE INVENTION

The present invention provides a method and apparatus for measuring analytes including, but not limited to, glucose, urea, and cholesterol in the tissue of a subject using Stokes Raman spectroscopy. Raman spectroscopy, by generating a distinct spectrum for each analyte, can resolve the individual components of the complex mixture present in blood and/or tissue of a subject such as for example a human or an animal.

In one embodiment, the present invention provides a method for in vivo detection of an analyte present in blood. The method comprises the steps of illuminating a portion of a sterile matrix beneath a nail by passing radiation from an optical source through the nail into the sterile matrix, collecting optical radiation emitted by blood present in the illuminated portion of the sterile matrix, and analyzing the collected radiation to determine if a selected analyte is present.

In an alternative embodiment, a laminar structure is provided for use in the detection of analytes present in a sterile matrix under a nail. The laminar structure comprises an optically transparent window plate having a first side and a second side, and a gel or viscous liquid layer affixed to the first side of the window plate. The gel or viscous liquid layer has a refractive index approximately equal to the refractive index of the nail.

In another embodiment, an analytical system is provided for in vivo identification and quantification of an analyte in blood. The system comprises a holder that comprises a means for exerting pressure on a finger or toe inserted into it to induce pooling of blood in a sterile matrix under a nail on the finger or toe. The system also comprises means for directing an incident excitation light beam to the finger or toe and through the nail and for focusing the beam at a focal point within the sterile matrix. Also provided are collection optics for collecting light emitted from scattering interactions within the sterile matrix and an analyzer for quantifying the emitted light.

### BRIEF DESCRIPTION OF THE DRAWINGS

Other objects and advantages of the present invention will become apparent upon reading the detailed description of the invention and the appended claims provided below, and upon reference to the drawings, in which:

FIG. 1 is a schematic diagram showing the anatomy of a fingertip.

FIG. 2 is a cartoon representation of a fingertip showing the contrast between the color intensity of a fingernail in its natural state (a) and with blood pooling resulting from pressure applied to the bottom and/or top of the fingertip:

FIG. 3 is a schematic representation of a gel adapted fingernail window interface according to one embodiment of the present invention.

FIG. 4 is a schematic diagram showing suitable collection optics according to one embodiment of the present invention.

FIG. 5 is a schematic diagram showing an alternative version of the collection arrangement of FIG. 4 according to another embodiment of the present invention.

FIG. 6 is a schematic diagram showing an alternative non-invasive probe configuration for detecting an analyte such as for example glucose under the nail according to another embodiment of the present invention.

FIG. 7 is a schematic diagram showing yet another system according to another alternative embodiment of the present invention.

FIG. 8 is a schematic diagram showing a design of a finger holder for Stokes Raman or other spectroscopy of blood in a sterile matrix according to one embodiment of the present invention.

FIG. 9 is a schematic diagram showing (a) a side view, (b) a top view, and (c) an “in use” view of a disposable form of a gel adapted window plate according to one embodiment of the present invention.

FIG. 10 is a chart showing the Stokes Raman spectra of whole blood and glucose.

FIG. 11 is a chart showing the Stokes Raman spectra of glucose and fingernail material.

FIG. 12 is a schematic diagram showing an optical arrangement using an optical coherence tomography (OCT) device for blood analyte detection through the finger nail according to one embodiment of the present invention.

FIG. 13 is a schematic diagram showing an optical arrangement for a reflective absorption spectroscopic device for blood analyte detection through the finger nail according to an alternative embodiment of the present invention.

### DETAILED DESCRIPTION OF THE INVENTION

In general, the present invention provides an optically based, non-invasive method and apparatus for the measurement of analytes, especially glucose, found in human blood. The method and apparatus may be used to alleviate the painful process of drawing blood, and allows repeated, accurate, reproducible testing of blood analyte levels. The method, which may employ Stokes Raman spectroscopy or other suitable methods of spectroscopy, utilizes the fingernail (or toenail) as a window into the human vascular system. The descriptions provided in this specification refer to Raman spectroscopy to illustrate one demonstrative embodiment of the present invention. However, one of ordinary skill in the art will realize, after reading the teachings provided herein, that the scope of the present invention encompasses the use of other spectroscopic methods as well.

By using the fingernail as the window, as opposed to the skin, the optical probe signal does not have to travel through the skin to excite the blood sample, nor does the Raman signal emitted by the blood sample have to travel back out through the skin to be measured. This eliminates or reduces the variability in signal strength and signal integrity from person to person based on ethnicity, physical condition, and/or environment, all of which can strongly affect optical transmission though the skin. The fingernail typically remains substantially independent of variations between individuals irrespective of their weight, race, profession, or most other variables.

The fingernail also provides good transparency to light in the visible and near-infrared regions of the electromagnetic spectrum. Given that signal collection is critical to a measurement's success, this transparency provides a significant advantage over other spectroscopic methods for measuring blood analyte concentrations which probe other parts of the body rather than the blood underneath the fingernail, or which require removal of a blood sample for in vitro analysis. Very few human tissues are transparent. Although the vitreous humor and aqueous humor in the eyeball are both transparent, as is necessary for human vision, the eyeball has poor blood circulation and a laser beam can easily damage the retina. In the present invention, the fingernail (or toenail) is used as a window to optically probe the tissue under the nail, which is called the sterile matrix.

Although the present invention will be discussed primarily in the context of glucose analysis, one of ordinary skill in the art should readily understand based on the descriptions and teachings provided herein that the scope of the invention also encompasses the detection of other blood components whose presence and/or concentration is relevant to medical diagnostics. In general, the method of the present invention comprises contacting tissue of the subject with excitation electromagnetic radiation having a wavelength in the range of approximately 400 nm to 2200 nm, alternatively in the visible blue to near IR range (about 400 nm to about 1000 nm) or about 600 nm to 980 nm (red to NIR). In one general embodiment, this analysis is performed while the tissue of the subject is in a blood replete state. In these ranges most blood constituents (and human tissue) show relatively little absorption, and hence a stronger Raman scattering. Examples of lasers suitable for use in producing the above-indicated excitation wavelength include, but are not limited to, external cavity diode lasers, gas lasers (HeNe, Argon ion, Krypton ion, or others) and semiconductor lasers. Suitable lasers, which emit in the above-indicated wavelength ranges are commercially available. Either pulsed or continuous wave (CW) lasers are suitable, although the latter is preferred. Use of a CW laser operating at a fixed wavelength in the above-indicated range has been found to be particularly advantageous.

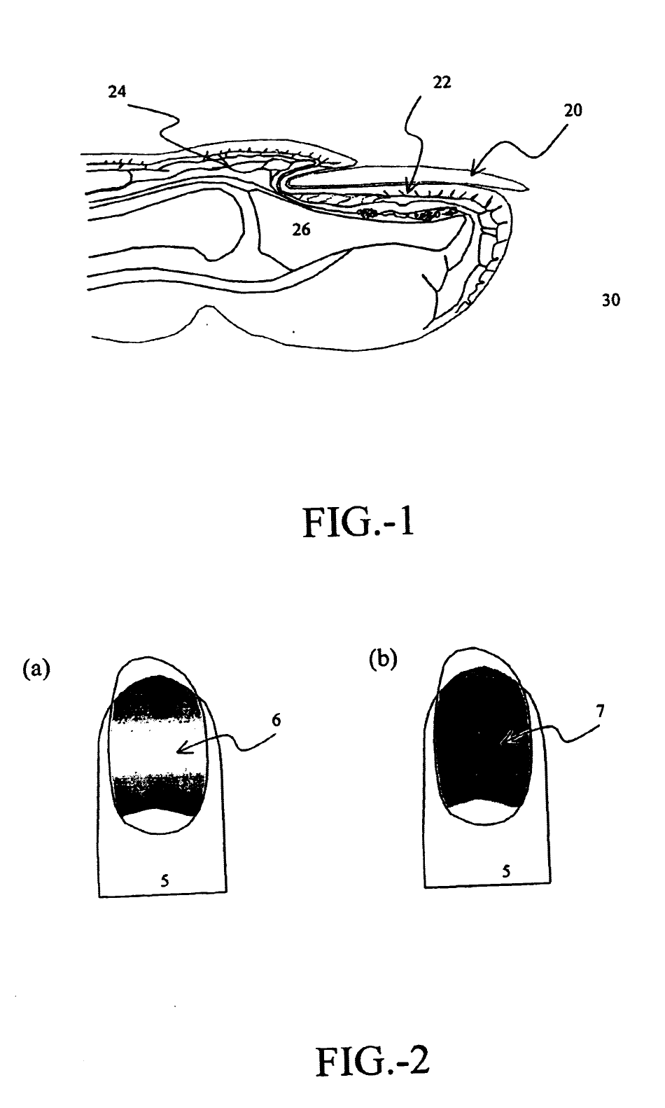

Some of the components of the fingertip 30 are shown in FIG. 1 which shows the general anatomy of a fingertip 30 including the nail plate 20, the sterile matrix 22, the Papilar network 24, and the fingertip bone 26. The sterile matrix 22 has a high density of blood capillaries and is therefore an ideal target tissue. When a fingertip 30 presses down, the sterile matrix 22 becomes blood replete and its color changes to appear dark red as a result of the blood pooling effect under the fingernail. As illustrated in FIG. 2, a fingernail in its natural state (FIG. 2a) exhibits a lighter color 32 compared to the darkening 34 resulting from blood pooling in the sterile matrix (the blood replete state) when pressure is applied to the bottom and/or top of the fingertip (FIG. 2b). This blood pooling in the sterile matrix 22 under the fingernail is advantageous in that it provides a high density of blood for Stokes Raman (or other optical) detection. The bottom surface of the nail 20 is directly connected to the sterile matrix 22, which is filled with blood, so that it can be considered as a part of the target tissue. The fingernail 20 itself is relatively transparent but its upper surface is frequently rough with grooves and/or other irregularities. Such a rough surface may cause problems because it tends to diffract and scatter incident light.

In one embodiment, the present invention addresses this problem by interfacing the upper surface of the fingernail to a smooth (i.e., flat and substantially optically transparent) surface (“window plate”) so as to allow the light to reach the tissue under the fingernail without significant scattering or distortion. To reduce scattering, a gel (or viscous liquid) having a refractive index which approximates the refractive index of the fingernail (about 1.5) fills the region between the rough surface of the nail and a glass (or other optically transparent material) window plate. In the case, for example, that the nail has a refractive index of 1.51, one can choose a gel also having a refractive index of 1.51, for example, NyoGel OCK-451 (Nye, Fairhaven, MA02719). By matching the refractive index of the gel to the refractive index of the fingernail, the refractive effect of the interface between the irregular nail surface and the gel on radiation passing through the interface is minimized. With this arrangement, light passes through the window plate, gel layer, and fingernail without significant refraction, reflection or scattering from the nail to gel or gel to window interface. Therefore, the laser can be focused down to the sterile matrix without undue interface loss or distortion. Also, the lens can image the Raman scattered radiation from the laser excited spot under the nail onto another object, such as a pinhole, optical fiber, fiber bundle, or spectrometer. The window plate will advantageously have an anti-reflection (AR) coating on its top surface (i.e. the surface facing away from the nail and toward the laser). However, it is not always necessary to have such an AR coating, because the window top surface causes only a small reflection loss, for example of approximately 4% per pass, and does not significantly scatter light or degrade the imaging properties of the optical system.

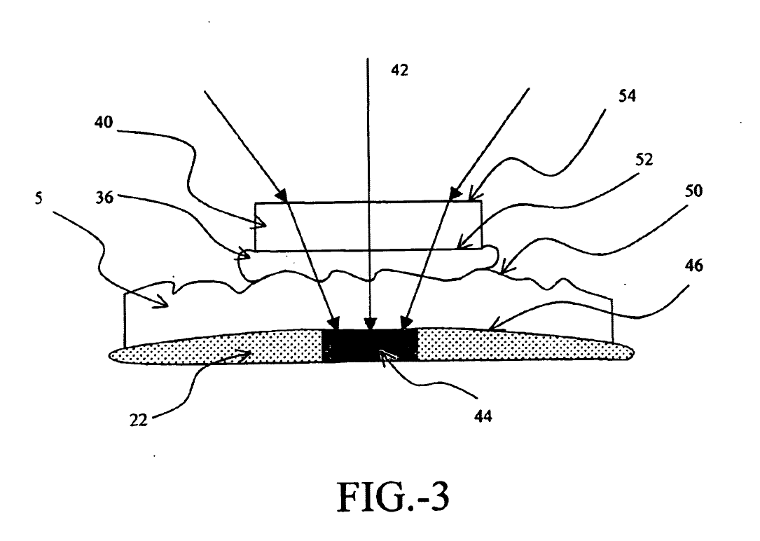

As illustrated in FIG. 3, the gel or viscous liquid 36 smoothes out any roughness on the surface 50 of the nail plate 20 by forming a seamless interface or a homogeneous optical surface between the gel 36 and nail 20. The gel 36 may advantageously be selected to have an index of refraction which is equal, or approximately equal, to the refractive index of the nail 20 thereby providing a homogeneous optical surface. In such a case, the gel 36 and nail 20 effectively become a single optical medium without any apparent interface between them. An optically transparent material, the window plate 40, having two substantially optically flat parallel surfaces 52, 54 is then placed on top of the gel 36 thereby forming a laminar structure which is substantially optically homogeneous. The window plate 40 material may advantageously be optical glass, plastic or other similar, optically transparent (in the indicated wavelength) material known in the art. The window plate 40, like the gel 36, will advantageously have a refractive index, which is close to that of the nail 20 (about 1.5). Laser light rays 42 which have a wavelength in the range of approximately 400 to about 2200 nm, are directed at the plate, pass through the plate 40, gel 36, and nail 20 without significant reflection or refraction until reaching the sterile matrix 22 under the nail 20. The sample volume 44 in FIG. 3 is defined by the location where Raman scattered radiation is generated by the rays impinging on the sterile matrix 22, which is filled with blood pooled by pressing the finger down. The nail 20 and the underlying sterile matrix 22 are generally joined by an interface 46 which is also optically transparent.

Use of the gel-adapted window on the nail 20, such as is shown in FIG. 3, produces several benefits. First, the focusing of the excitation laser beam onto the blood sample in the sterile matrix 22 is improved relative to a situation where surface roughness of the nail 20 causes scattering of the incident light. The excitation power is more concentrated on the sample volume 44, containing for example, glucose, so that less power is needed from the laser. Second, Raman scattered radiation emitted from the sampled tissue 44 experiences reduced loss and distortion. The reduced distortion allows the Raman scattered radiation to be imaged into collection and detection optics with improved performance (i.e., greater efficiency). If imaging optics are used, they can advantageously provides spatial filtering to help remove other emitted radiation (such as fluorescence and elastic scattering).

An alternative to the use of a gel/nail interface is the use of a fingernail polish type coating with a nail matching refractive index to fill the rough surface or interstices of the finger nail to provide a smooth surface toward the incident radiation. Another alternative is to clean and polish (i.e., smooth) the nail surface. In some cases, (e.g., the thin smooth nail of a baby), there may be no need for any these methods for reducing the effect of scattering and distortion introduce by a rough nail-air interface.

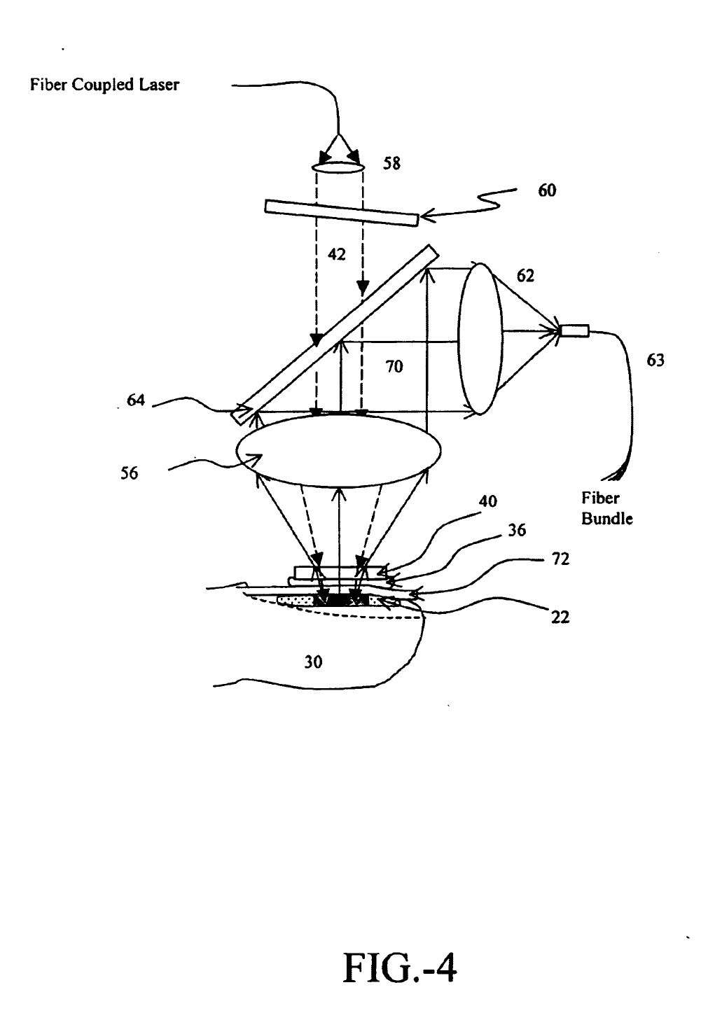

A system for spectroscopically analyzing tissue under a nail according to one embodiment of the present invention is illustrated in FIG. 4 which generally shows an excitation laser beam focused onto the sterile matrix and part of the collection optics for the resulting Raman scattered radiation. The excitation laser has a wavelength that may advantageously be approximately 830 nm in the near IR. As noted above, one of ordinary skill in the art will understand that other wavelengths may be used based on routine experimentation using the teachings provided herein. The laser beam is passed through a dichroic beam splitter having high transmission. Raman light collected from the sterile matrix is reflected by the beam splitter because it is at a different wavelength from the incident laser light. The reflected Raman scattered light is then coupled into a spectrometer to record the Raman spectrum.

Referring more specifically to FIG. 4, incident light 66 (dashed arrows) from a fiber-coupled laser (not shown) is collimated using a lens 58 and sent through a narrow bandpass filter 60 to ensure spectral purity. The laser beam 42 then passes through a beam splitter 64 and is focused by a lens 56 through the gel 36 adapted window 40 and fingernail 20 onto the sterile matrix 22. The Raman scattered light (solid arrows) 70 is collected by the lens 56 and reflected by splitter 64 to take a route different from that of the incident light. This Raman scattered light is focused by a second lens 62 into a fiber bundle 63, which delivers the light to a spectrometer (not shown).

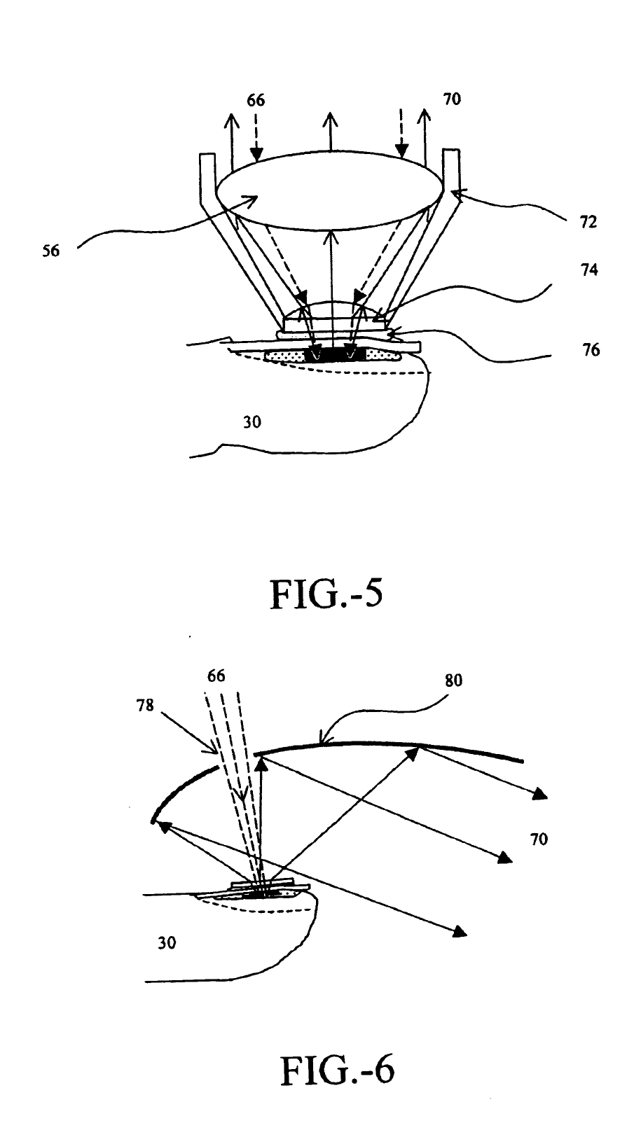

Another embodiment of a system according to the present invention is illustrated in FIG. 5. In general, the flat window 40 of FIG. 4 is replaced by an objective lens 74, to enhance the collection of incident light having a high divergent angle. Second, a viscous index matching liquid 76 with a refractive index matched to that of the nail, approximately 1.51, may replace the gel 36 used in previously described embodiments. The index matching liquid 22 has sufficient mobility to allow relative motion between the objective lens and the fingernail. Third, the objective lens 74 is joined onto the main lens via a lens holder 72 so that the combination provides a lens system. As in FIG. 4, the incident excitation radiation 66 (dashed lines) is focused by a lens 56. Emitted Raman light 70 (solid lines) passes in turn back out of the lens 56 to be collected and analyzed.

In another embodiment, shown in FIG. 6, an off-axis parabolic mirror 80 may be substituted for the first collecting lens 56 shown in FIG. 4. A parabolic mirror can provide a higher numerical aperture (NA) for improved light collection. As shown generally in FIG. 6, the excitation laser beam 10 may be focused through a small hole 78 in a parabolic mirror 80 and then through a gel-adapted window such as has been described above and then finally through the fingernail 20 to excite a blood sample present in the sterile matrix 22. The Raman scattered light 70 coming out of the window from the blood sample may be collected by the parabolic mirror 80 and directed to a spectrometer (not shown).

Under the nail 20, a sample volume of blood within the sterile matrix 22 is defined by the focal diameter and focal depth of the collecting optics. In the sterile matrix tissue, Raman radiation is emitted from a sample volume 44, as illustrated in FIG. 3. The laser beam spot generally becomes more diffuse than the ideal Gaussian beam waist as it penetrates tissue, due to elastic scattering. Since Raman scattered radiation is isotropic relative to the incident radiation, Raman radiation power is proportional to the excitation power but does not depend strongly on the incident laser beam direction. Raman light from this sample volume is advantageously imaged into a multimode fiber or a bundle of fibers. An imaging optical system provides the opportunity to spatially filter the signal to facilitate noise reduction.

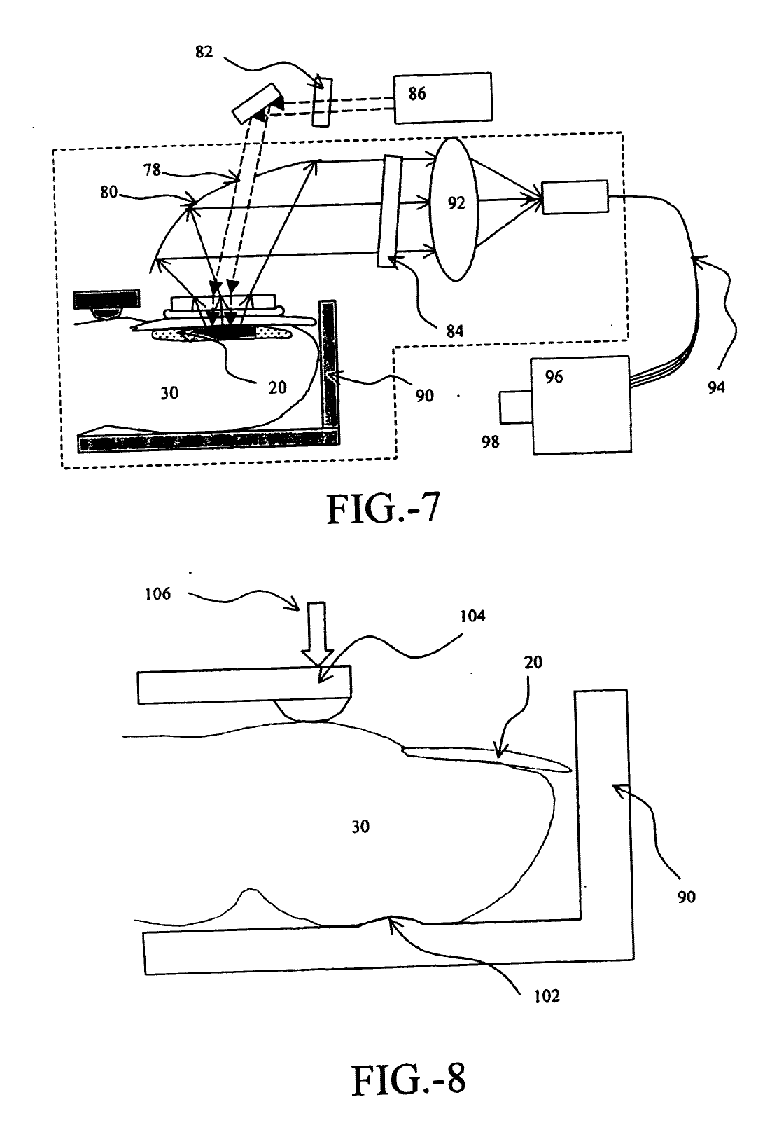

FIG. 7 shows another embodiment of the invention including additional aspects of the invention, including a near infra-red laser for illumination and a holographic grating based spectrometer to record the Raman signal as a function of wavelength. The near infra-red (NIR) light may be delivered via a single mode fiber from a frequency stabilized laser diode with a wavelength of, for example, approximately 830 nm. Use of this wavelength is advantageous because current commercial silicon charge coupled device (CCD) arrays are responsive to the resulting Raman radiation wavelengths. A further advantage arises from the tendency of 830 nm radiation to not excite the fluorescence of human tissue as strongly as visible light. The laser beam may be filtered using a band pass filter to ensure side mode suppression and to remove or reduce any extraneous laser noise. The light may be delivered to an enclosure around the finger. The nail bed may be illuminated through a gel or viscous liquid that is index matched to the index of refraction of the fingernail. The Raman scattered radiation emitted by the illuminated sterile matrix under the fingernail may be collected using an off-axis parabolic mirror that may be advantageously directed to a multi-mode fiber bundle where the light may be further filtered to suppress any remaining pump light. The multi-mode fiber bundle may be matched to the etendue (area-solid angle product) of a large numerical aperture, holographic grating based spectrometer, where the signal will be dispersed. The dispersed signal may be read by a CCD array with high quantum efficiency in the near infra-red. The CCD array may be interfaced to a computer that provides data logging and data analysis capability. In order to optimize analysis results, system noise and background noise may be subtracted off from the raw spectral signal provided by the CCD array using known techniques. In addition, fluorescence from human tissue fluorophores may be fitted with a high order polynomial and may also be subtracted off. The remaining Raman signatures may be used in a calibration process and the analyte concentrations determined using a partial least squares algorithm or other suitable multivariate regression analysis technique known in the art. The above-indicated analysis techniques are described in, for example, “Multivariate Calibration” by H. Martens and L. Tormod Naes, John Wiley & Sons, 1089 ISBN o-471-90979-3; Partial Least-Squares for Spectral Analyses, 1, by D. Haaland and E. V. Thomas Anal. Chem. 60, 1193-1202 (1988); and Partial Least-Squares Regression; A Tutorial, by P. Geladi and B. Kowalski Analytica Chimica Acta, 185 (1986) pages 1-17, the disclosures of which are incorporated herein by this reference.

More specifically, as shown in FIG. 7, a beam of, for example, 830 nm light from a diode laser 86 is passed through a bandpass filter 82 and then passed through a parabolic mirror 80 by means of a small hole 78 in the mirror, and is focused onto a gel window adapted nail 20, under which a blood sample from the blood rich capillaries in the sterile matrix is pooled under pressure. Raman-scattered light emitted from blood in the sterile matrix (typically having ˜1 mm² area) is collected by the mirror 80, passed through a notch filter 84 configured to reject 830 nm light, and then focused by a lens 92 into an optical fiber bundle 94, which converts the circular shape of the collected light to a rectangular shape to match the entrance slit of a spectrograph 96. The spectra are collected by a cooled charge coupled device (CCD) array detector 98 (e.g., one having 1024×256 pixels) and binned along the vertical direction, resulting in an 1024 pixel spectrum.

Although the patient may simply press his/her finger down on a flat surface to cause the sterile matrix to become blood replete, use of suitable clamp means to apply downward pressure and maintain the finger stationary is advantageous. One representative example of a finger holder suitable for use with the invention, which comprises a base and a clamp, is shown in FIG. 8. After inserting the finger into the holder 90, the fingertip rests on the base and touches a bump, 102, that may be present on the upper surface of the base, which pushes the finger up. A clamp 104 presses down (force vector 34) and also tends to hold the finger in place with a touch pad having, for example, a half round shape. The touch pad is preferably of a resilient material that does not discomfort the finger but still applies sufficient pressure to hold it stationary. This arrangement can be adjusted to provide a level of force on the fingertip that provides the maximal amount of blood pooling in the sterile matrix. A suitable pressure will generally range from about 1 to about 4 Newtons. The pressure from both top and bottom will temporarily suppress the digital vascular blood flow, thereby causing the sterile matrix to be in the blood replete state. As a result, there will be increased blood pooling under the nail. When the sterile matrix is in the blood replete state the color under the nail will appear red to dark red such as is illustrated schematically in FIG. 2b. During the blood pooling, the pulse caused fluctuation is also minimized. The holder of FIG. 8 provides enhanced and steadier blood pooling than simply pressing the finger down. Therefore, such a finger holder not only holds the finger in place, but also creates an ideal situation for blood pooling. After clamping down, the finger holder may, if desired, be traversed to optimize the alignment of the fingernail sterile matrix with the focus of the laser beam and the focus of the parabolic mirror. Alternatively, the illumination and collection optical system may be translated instead of moving the finger holder, which may remain stationary.

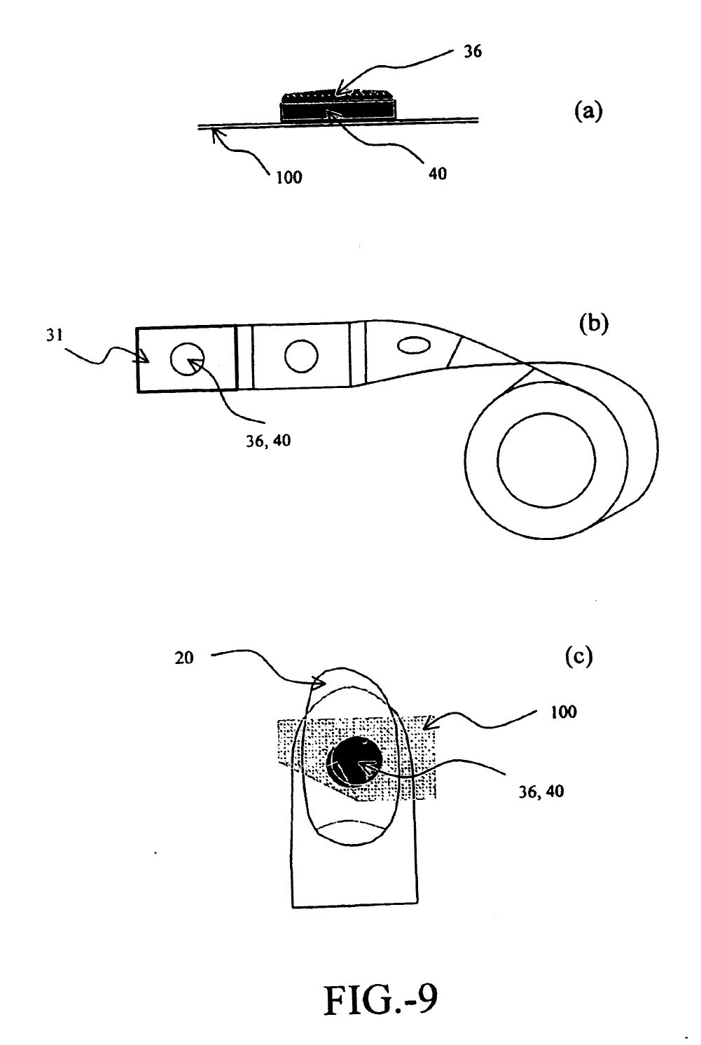

An advantageous form of a gel-adapted window, called a “gel adapted window sticker,” according to one embodiment of the present invention is shown in FIG. 9a, FIG. 9b and FIG. 9c. As shown in FIG. 9, the window plate 40 which is attached to a piece of release paper 100 (or other suitable removable support material) and the other side of the window plate 40 is covered by a thin layer of gel 36. A plurality of individually separable window plates having a gel layer 36 on one side thereof can be affixed to a release paper strip with the gel side facing away from the paper 100 is shown in FIG. 9b. The paper may be held to place the gel-adapted window onto the fingernail gel side down. The gel coated plate may be applied to a nail by pressing it on and then peeling off the release paper. The paper strip 100 acts to protect the non-gel contacting surface of the window prior to use. FIG. 9c schematically shows the application of one of the gel adapted windows to a fingernail X20. After pressing the gel side of the window plate onto the fingernail, the support material 100 is peeled off. A touch on the top surface of the window plate 40 may mar the polished optical surface with finger-prints and/or other residues, which could degrade its optical performance. The release paper serves to protect this optical surface until the window is actually used. These gel-adapted windows may advantageously be disposable, which eliminates the need to consider methods for keeping the top surface of the window optically clean for extended periods of time. The gel adapted window sticker may be in the form of a continuous strip (which can be rolled up) with each individual unit (i.e., gel, window and release paper) being separable as shown in FIG. 9b or each unit may stand alone with its own cover sheet on the gel side.

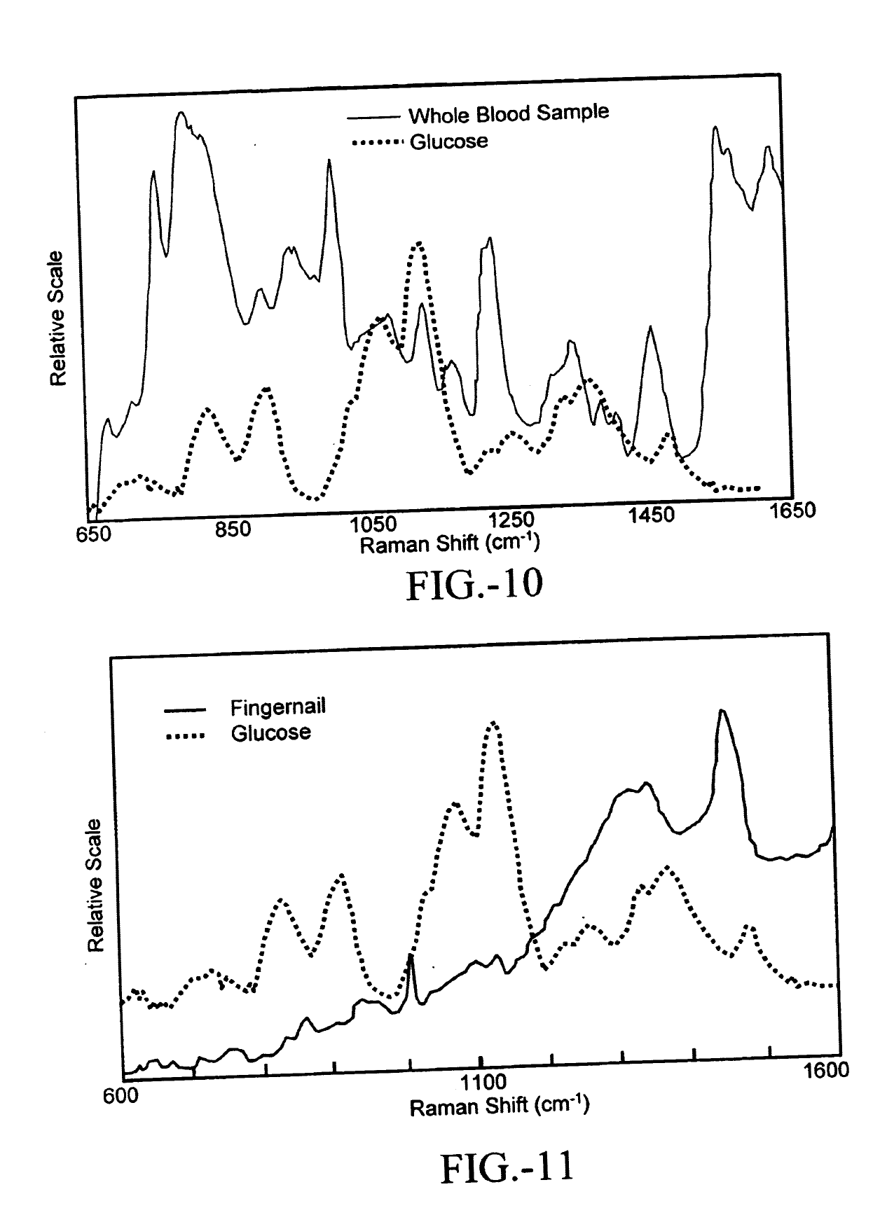

FIG. 10 and FIG. 11 show data comparing the Raman spectra of glucose to the Raman spectra of whole blood and to the Raman spectra of nail material to examine possible overlaps in the Raman spectrum. In FIG. 10 the glucose Raman spectrum is compared with the whole blood Raman spectra. The curve shown in FIG. 10 is after that shown by Annika M. K. Enejder, Tae-Woong Koo, Jeankun Oh, Martin Hunter, Slobodan Sasic, Michael Feld, and Gary L. Horowitz, “Blood analysis by Raman spectroscopy”, Optics Letters Vol 27, No. 22, 2004-2006, 2002. In FIG. 11 the glucose spectrum is compared with the fingernail Raman spectrum. The fingernail data shown in FIG. 11 follows Williams A C, Edwards H G M and Barry B W, “Raman Spectra of Human Keratotic Biopolymers: Skin, Callus, Hair and Nail”, J. Raman Spectr. V25, 95-98 (1994). In both figures, the glucose spectrum is readily distinguishable from either the whole blood spectrum or the fingernail spectrum. Thus Raman scattering from blood or from the fingernail does not preclude the detection of glucose by Raman scattering.

The above optical arrangement of the fingernail can be advantageously applied to other methods for optically probing the sterile matrix. Other optical probing/optical spectroscopy techniques will also benefit from the use of the fingernail as a window into the blood. The benefits are due to the fact that these techniques rely on the returning optical signal strength and quality to reveal information. Since the fingernail is substantially transparent in comparison to the skin, a significant benefit can be thereby realized.

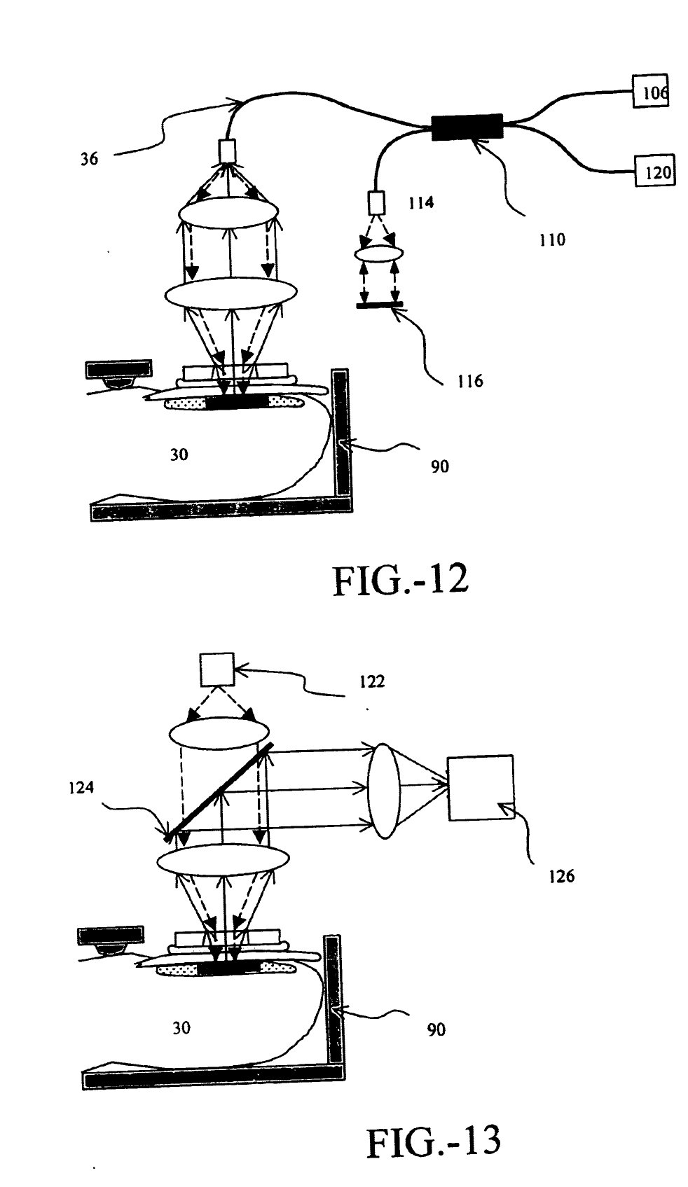

One such method is optical coherence tomography (OCT), which entails determining glucose or other analyte concentration by measuring the scattering loss differentiation in the tissue. OCT is a known analytical technique and is described, for example in Optics Letters Vol. 19, No. 8 Apr. 15, 1994 pages 590-592 and Phys. Med. Biol. 48 (2003) pages 1371-1390. The teaching of both these references is incorporated herein. The optical source for the OCT system is generally an incoherent source having a broad band spectrum (e.g., as provided by a light emitting diode, incandescent lamp, or superluminescent diode). In FIG. 12, the broad band light source 106 first passes through a beam splitter 110 which has approximately 50% transmission over the whole spectral region. The light from fiber arm 112 is collimated and then focused through a gel-adapted window and fingernail without suffering significant loss from scattering or reflection, as discussed above. The incident light beam, when focused onto the sterile matrix, interacts with a sample volume of blood within the sterile matrix. Light reflected by the sample volume is collimated by a lens and directed back to the fiber arm 112. After passing through the splitter 110, it reaches the detector 120. The other fiber arm 114 of the splitter 110 is sent to a translation scanning mirror 38, the reflected light is sent into the fiber arm 114. It passes through splitter 110 to detector 120 to interfere with light from fiber arm 36 as described above. By varying the length of the interferometer arm 116, the signal due to emission from various depths within the sample volume may be determined. This depth-specific signal is accomplished by using the inherently limited coherence length of the broadband source. Only signals from the tissue that are coherent with the retro-reflected signal will mix coherently at the detector. The coherently mixed signal is thus preferentially detected.

OCT has been used previously with limited success for imaging human tissues through the skin. For glucose detection, it is based on measuring scattering loss variation in the dermis caused by the intercellular fluid index change. The intercellular fluid index is significantly changed by a change in glucose concentration. In prior art applications of this technique, the probing light beam encounters serious problems induced by scattering losses in the epidermis. These losses reduce the signal strength and induce signal echoes. Consequently, noise and artificial peaks/valleys are introduced to the scattering loss curve. In the present invention, the use of a gel-adapted window on the fingernail provides an optical window directly into the target tissue, in this case the sterile matrix under the nail. Because of this clear window, the probe beam and emitted radiation experience minimal loss and scattering so that more light may be coupled to the interferometer to thereby provide a stronger OCT signal. The clear window generally introduce little echo or distortion to the light beam. As a result, the OCT scattering loss curve may be greatly improved. In addition, as previously indicated, the sterile matrix under the fingernail is filled with a dense capillary vascular network, which is finely distributed with greater uniformity than in other locations, thus providing an optimal probe location.

Another analytical method that may benefit from use of the nail as a window is NIR reflective or absorption spectroscopy where the collected light is dispersed with a spectrograph. This technique is described in Optics Letters Vol. 19, No. 24, Dec. 15, 1994, pages 2062-2064. FIG. 13, illustrates a broadband light source 122 which passes through a beam splitter 124 and through the nail 20 to illuminate the sterile matrix 22. In the sterile matrix 22, a number of substances, such as water, glucose, and other compounds having O—H and/or N—H groups will have certain absorption peaks in the NIR region of the electromagnetic spectrum due to interactions of the overtone vibrations of these groups. The reflected light from the sterile matrix is collected by a lens through beam splitter 124 and projected onto the detector 126. The detector may include a spectral dispersing device such as a grating to record the spectrum. From spectral fitting of such a spectrum, the glucose concentration may be determined. This method is essentially absorption spectroscopy making use of back-reflected and/or elastically scattered light from the sample. The spectral fitting methods may be artificial neural networks, or partially least square fit. This method provides a number of advantages over previous applications of reflective absorption spectroscopy to in vivo detection. Previously, in reflective absorption spectroscopy, the light has been passed through the skin of the forearm, fingertip or other outside the body location. All such locations have drawbacks. First, skin causes scattering and absorption loss for both the incident beam and also for radiation emitted from the sample volume, which complicates the analysis and interpretation of the measured spectra of the target tissue. Second, most other locations do not have the high blood concentration provided by the dense capillary vascular network found in the sterile matrix. The present invention provides an ideal optical window to allow the light to directly reach the target tissue, namely the sterile matrix under a fingernail. As a result, there is far less intermediate influence on the target spectrum. Furthermore, the blood pooled sterile matrix provides more blood, which affords a stronger signal.

The foregoing description of specific embodiments and examples of the invention have been presented for the purpose of illustration and description, and although the invention has been illustrated by certain of the preceding examples, it is not to be construed as being limited thereby. They are not intended to be exhaustive or to limit the invention to the precise forms disclosed, and obviously many modifications, embodiments, and variations are possible in light of the above teaching. It is intended that the scope of the invention encompass the generic area as herein disclosed, and by the claims appended hereto and their equivalents.

1. A method for in vivo detection of an analyte present in blood, comprising the steps of:
illuminating a portion of a sterile matrix beneath a nail by passing radiation from an optical source through the nail into the sterile matrix;
collecting optical radiation emitted by blood present in the illuminated portion of the sterile matrix; and
analyzing the collected radiation to determine if a selected analyte is present.

2. The method of claim 1, wherein the analyte is selected from the group consisting of glucose, urea, cholesterol, triglycerides, total protein, albumin, hemoglobin, hematocrit, and bilirubin

3. The method of claim 2, wherein the analyte is selected from the group consisting of glucose, urea, and cholesterol.

4. The method of claim 3, wherein the analyte is glucose.

5. The method of claim 1, wherein the nail is a fingernail.

6. The method of claim 1, wherein the illuminating radiation has a wavelength in the range of approximately 400 nm to 2200 nm.

7. The method of claim 1, wherein the optical source is a CW laser and the radiation has a wavelength in the range of approximately 600 nm to 900 nm.

8. The method of claim 1, wherein the optical source is a laser operating at a fixed wavelength, and the collected radiation comprises Stokes Raman radiation.

9. The method of claim 1, further comprising the step of:
interposing between the optical source and the nail a window plate and a gel or viscous liquid having a refractive index that is approximately equal to the refractive index of the nail, the gel or viscous liquid forming a homogenous optical surface with the nail and the window plate being in direct contact with the surface of the gel or viscous liquid distal from said nail.

10. The method of claim 9, wherein the window plate has a refractive index that is approximately equal to the refractive index of the nail.

11. The method of claim 1, wherein the radiation is analyzed by multi-variate regression analysis

12. The method of claim 9, wherein the nail is a fingernail.

13. The method of claim 1, wherein the sterile matrix is caused to be in a blood replete state by applying a pressure of from about one to about four Newtons to the top of a finger of which the sterile matrix forms a part.

14. The method of claim 1, wherein the source radiation is multi-wavelength radiation, and the collected radiation is analyzed by reflection absorption spectroscopy.

15. The method of claim 1, wherein the source radiation is multi-wavelength radiation, and the collected radiation is analyzed by optical coherence tomography.

16. A laminar structure for use in the detection of analytes present in a sterile matrix under a nail, comprising:
an optically transparent window plate having a first side and a second side, and
a gel or viscous liquid layer affixed to the first side of the window plate, the gel or viscous liquid layer having a refractive index approximately equal to the refractive index of the nail.

17. The structure of claim 16, wherein the window plate has a refractive index approximately equal to the refractive index of the nail.

18. The structure of claim 16 further comprising a film releaseably affixed to the second side of the window plate.

19. A plurality of the structures of claim 16 separably affixed to each other in the form of a continuous strip.

20. An analytical system for in vivo identification and quantification of an analyte in blood, comprising:
a holder, the holder comprising a means for exerting pressure on a finger or toe inserted into the holder to induce pooling of blood in a sterile matrix under a nail on the finger or toe;
means for directing an incident excitation light beam to the finger or toe and through the nail and for focusing the beam at a focal point within the sterile matrix; and
collection optics for collecting light emitted from scattering interactions within the sterile matrix; and
an analyzer for quantifying the emitted light.
